(19) United States
(12) Patent Application Publication
(10) Pub. No.: US 2026/0116852 A1
(43) Pub. Date: Apr. 30, 2026

(54) ß-CAROTENE SYNTHESIS (II)

(71) Applicant: DSM IP ASSETS B.V., Maastricht (NL)

(72) Inventors: Werner BONRATH, Kaiseraugst (CH); Patrick FREY, Kaiseraugst (CH); Leonardo LE, Kaiseraugst (CH); Christian LOTHSCHUETZ, Kaiseraugst (CH); Bettina WUESTENBERG, Kaiseraugst (CH)

(21) Appl. No.: 19/115,488

(22) PCT Filed: Sep. 25, 2023
(86) PCT No.: PCT/EP2023/076343

§ 371 Date: Mar. 26, 2025

(30) Foreign Application Priority Data
Sep. 29, 2022 (EP) 22020468.9

(51) Int. Cl. C07C 403/24

(52) U.S. Cl. CPC C07C 403/24; C07C 2601/16

(57) ABSTRACT

The present invention relates to process for producing β-carotene in a specific solvent.

The present invention relates to process for producing β-carotene in a specific solvent.

β-Carotene is an organic, strongly coloured red-orange pigment abundant in fungi, plants, and fruits. β-Carotene is an important product with many different ways of application. β-Carotene is the compound of the following formula (I)

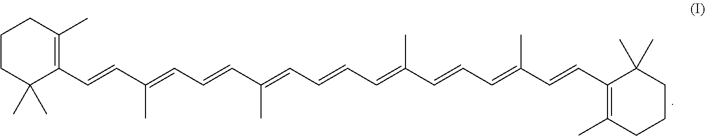 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

β-Carotene is a member of the carotenes, which are terpenoids (isoprenoids), synthesized biochemically from eight isoprene units and thus having 40 carbons. Among the carotenes, β-carotene is distinguished by having beta-rings at both ends of the molecule.

β-Carotene is the most common form of carotene found in plants.

When used as a food colouring, it has the E number E160a (ii).

Furthermore, in nature, β-carotene is a precursor (inactive form) to vitamin A via the action of beta-carotene 15,15′-monooxygenase.

β-Carotene is a compound that gives vivid yellow, orange, and red colouring to vegetables. The human body converts β-carotene into vitamin A (retinol).

Next to its dyeing properties β-carotene has also some health benefits, such as effects on eye health, on improved cognitive function, on skin protection and on cancer prevention.

A common way to produce β-carotene is shown in the following scheme

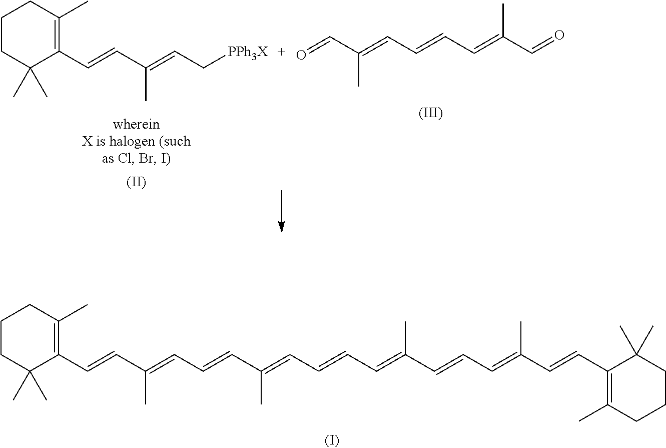

Due to its importance, there is always a need for an improved way to obtain β-carotene.
Surprisingly, we found that the choice of a specific solvent leads to an excellent yield and excellent purity of the resulting β-carotene.

This specific solvent is an alcohol represented by the compound of formula (IV)

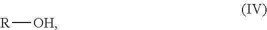

wherein
R is a linear or branched C₃-C₁₀-alkyl moiety.

Therefore, the present invention relates to the process (P) for the production of the compound of formula (I)

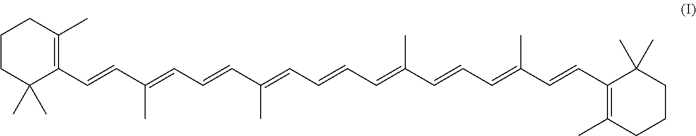 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

wherein
a compound of formula (II)

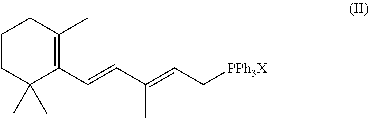

wherein
X is halogen (preferably Cl, Br or I)
is reacted with a compound of formula (III)

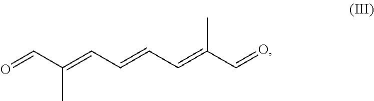

characterised in that at least one compound of formula (IV)

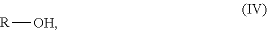

wherein
R is a linear or branched C₃-C₁₀-alkyl moiety
is used as the solvent.

In the context of the present invention, all disclosed compounds (represented by the chemical formulae) can be in any possible stereochemical configuration.

The process according to the present invention has some advantages.

One major advantage of the present improved synthesis lies therein that the solvent (or the mixture of solvents) used is halogen-free and non-aromatic. This means that the solvent used is eco-friendly. Furthermore, the solvents used in the new process according to the present invention can be regenerated easily. When using ethanol (as in many of the prior art processes) such a regeneration is not so easy to be done.

Preferably, a compound of formula (II),
wherein
X is Cl, Br or I,
is used.

More preferably, the compound of formula (IIa)

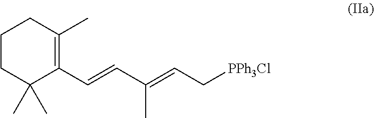

is used.

Therefore, the present invention relates to a process (P1), which is process (P), wherein a compound of formula (II) wherein X is Cl, Br or I.
is used.

Therefore, the present invention relates to a process (P1′), which is process (P), wherein a compound of formula (IIa)

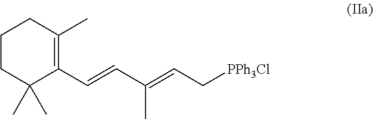

is used.

The process according to the present invention is carried out in at least one alcohol of formula (IV) as solvent.

Preferably, the process according to the present invention is carried out in at least compound of formula (IV)
wherein
R is a linear or branched C₄-C₈-alkyl moiety.

More preferably, the process according to the present invention is carried out in at least compound of formula (IV), wherein
R is a linear or branched C₄-C₇-alkyl moiety.

Especially preferred are compounds of formula (IV), wherein R is a linear or branched C₅alkyl moiety. 10 Most preferably, 1-pentanol, 2-pentanol, 2-methyl-butan-1-ol, 2-methyl-2-butanol and/or 3-methyl-1-butanol is used as solvent.

Even more preferred is a mixture of 2-methyl-2-butanol and 3-methyl-1-butanol. Most preferred is a 25:75 (in relation to wt-% of the solvents, based on the total weight of the solvent mixture) of 2-methyl-2-butanol and 3-methyl-1-butanol.

Therefore, the present invention relates to a process (P2), which is process (P), (P1) or (P1′), wherein the process is carried out in at least one alcohol of formula (IV), wherein R is a linear or branched C₄-C₈-alkyl moiety.

Therefore, the present invention relates to a process (P2′), which is process (P), (P1) or (P1′), wherein the process is carried out in at least one alcohol of formula (IV), wherein R is a linear or branched C₄-C₇-alkyl moiety.

Therefore, the present invention relates to a process (P2″), which is process (P), (P1) or (P1′), wherein the process is carried out in at least one alcohol of formula (IV), wherein R is a linear or branched C₅-alkyl moiety.

Therefore, the present invention relates to a process (P2′″), which is process (P), (P1) or (P1′), wherein the process is carried out in 1-pentanol, 2-pentanol, 2-methyl-butan-1-ol, 2-methyl-2-butanol and/or 3-methyl-1-butanol.

Therefore, the present invention relates to a process (P2″″), which is process (P), (P1) or (P1′), wherein the process is carried out in a mixture of 2-methyl-2-butanol and 3-methyl-1-butanol.

Therefore, the present invention relates to a process (P2′″″), which is process (P), (P1) or (P1′), wherein the process is carried out in a 25:75 (in relation to wt-% of the solvents, based on the total weight of the solvent mixture) of 2-methyl-2-butanol and 3-methyl-1-butanol.

Usually, a base is added to the reaction mixture as well.

The base is usually an alkali hydroxide, earth alkali hydroxide, alkali carbonate, earth alkali carbonate, KF/Al₂O₃, NaOCH₃ and KOCH₃.

Preferably, the base is CsOH, KOH, NaOH, Na₂CO₃ K₂CO₃ or Na₂CO₃

The base is usually used in molar excess in view of the compound of formula (III). Usually 2-20 mol-equivalent in view of the compound of formula (III).

Therefore, the present invention relates to a process (P3), which is process (P), (P1), (P1′), (P2), (P2′), (P2″), (P2′″), (P2″″) or (P2′″″), wherein the process is carried out in the presence of at least one base.

Therefore, the present invention relates to a process (P3′), which is process (P3), wherein the base is chosen from the group consisting of alkali hydroxide, earth alkali hydroxide, alkali carbonate, earth alkali carbonate, KF/Al₂O₃, NaOCH₃ and KOCH₃.

Therefore, the present invention relates to a process (P3″), which is process (P3), wherein the base is chosen from the group consisting of CsOH, KOH, NaOH, Na₂CO₃ and K₂CO₃.

The compound of formula (II) is used in an amount of at least 2 mol-equivalent (in view of the compound of formula (III).

Therefore, the present invention relates to a process (P4), which is process (P), (P1), (P1′), (P2), (P2′), (P2″), (P2′″), (P2″″), (P2′″″), (P3), (P3′) or (P3″), wherein the compound of formula (II) is used in an amount of at least 2 mol-equivalent (in view of the compound of formula (III).

The compound of formula (IV) is used as a solvent and is therefore used in excess with regard to the compound of formula (II) as well as compound of formula (III).

The base, which is usually and preferably used in the process according to the present invention is used in excess of the compound of formula (II).

Usually and preferably, it is used in an amount of 1.5 to 4 mol-equivalent (in view of the compound of formula (II).

Therefore, the present invention relates to a process (P5), which is process (P3), (P3′) or (P3″), wherein the at least one base is used in an amount of 1.5 to 4 mol-equivalent (in view of the compound of formula (II).

The process according to the present invention is usually carried out a temperature of −5° C. to 120° C.

Therefore, the present invention relates to a process (P6), which is process (P), (P1), (P1′), (P2), (P2′), (P2″), (P2′″), (P2″″), (P2′″″), (P3), (P3′), (P3″), (P4) or (P5), wherein process according to the present invention is usually carried out a temperature of −5° C. to 120° C.

The following Examples illustrate the invention further without limiting it. All percentages and parts, which are given, are related to the weight and the temperatures are given in ° C., and the pressures are absolute pressures when not otherwise stated.

### EXAMPLES

### Example 1

Under inert gas atmosphere, C₁₀-dialdehyde (0.53 g, 1 eq, 3.2 mmol), which is the compound of formula (III) and vinyl salt (5.0 g, 2.3 eq, 7.4 mmol), which is the compound of formula (IIa) were suspended in 1-pentanol (14.9 mL). The yellow suspension was warmed to 38° C. A sodium hydroxide solution (2.8 g, 2.6 mL, 10% wt, 2.2 eq, 7.1 mmol) was added dropwise over 16 h. Then the reaction mixture was stirred for another 1 h at 40° C. and 1 h at 100° C. After 30 min at 100° C., a color change from orange to violet indicated a change of crystal form. The oil bath was removed and the reaction mixture was cooled to 40° C. Then, 5 ml of deionized water were added and the reaction mixture was cooled to 20° C. The red suspension was filtered the filter cake was subsequently rinsed with pentanol and water. The red crystals were dried in vacuum at 50° C. overnight. The product was obtained as dark violet crystals (1.58 g) in 86% yield and 93.7% purity.

### Example 2

Under inert gas atmosphere, a solution of vinyl salt in pentanol (13.1 g, 25.9% wt, 2.1 eq, 6.75 mmol) was adjusted with an aqueous sodium hydroxide solution (5%) to neutral pH. To this solution was added C₁₀-dialdehyde (0.530 g, 1 eq, 3.21 mmol) and pentanol (5 mL). The yellow suspension was cooled to 0° C. A sodium hydroxide solution (2.3 g, 2.1 mL, 10% wt, 1.76 eq, 5.66 mmol) was added over 2 h. After that a second portion of sodium hydroxide solution (0.57 g, 0.51 mL, 10% wt, 0.44 eq, 1.41 mmol) was also added over 2 h. Then, the reaction mixture was warmed to 40° C. and stirred for 1 h, followed by heating to reflux for another 1 h. After 30 min, a color change from orange to violet indicated a change of crystal form. The oil bath was removed and the reaction mixture was cooled to 40° C. Deionized water (5 mL) was added and the reaction mixture was cooled to 0° C. The red suspension was filtered and the filter cake was subsequently rinsed with pentanol and water. The red crystals were dried in vacuum at 50° C. overnight.

The product was obtained as dark violet crystals (1.67 g) in 90% yield and 92.8% purity.

### Example 3

In a 1 liter reactor, C₁₀-dialdehyde (9.03 g), which is the compound of formula (III), and K₂CO₃ anhydrous (18.9 g) were loaded, and 3-methyl-1-butanol (97.5 g) was added. The mixture was heated to an internal temperature of 40° C. After this, vinylsalt (60.1 g), which is the compound of formula (IIa), dissolved in 156 g of 3-methyl-1-butanol was added (max conc. of water in this mixture must not exceed 2%-w/w). The addition was done over a period of 60 min.

After complete addition of the vinylsalt solution, the dark red suspension was stirred for 240 min at 40° C. At the end of this time, another 0.02 eq. of vinlysalt (as solution in 3-methy-1-butanol) were added.

The reaction mixture was heated to 90° C. The reaction mixture was kept at this temperature for another 2 h.

Finally, the reaction mixture was cooled to 20° C. and deionized water (150 g) was added.

The solids were filtered off.

The solid product was washed with MeOH (90.0 g), water (200 g) and finally again with MeOH (90.0 g). The dark violet solid was dried under reduced pressure at 40° C. for 8 hours.

Yield: 28.41 g of β-carotene crude as isomeric mixture of all-trans β-carotene (85-92%-w/w) and cis-isomers of β-carotene (8-15%-w/w).

1. Process for the production of to the compound of formula (I) 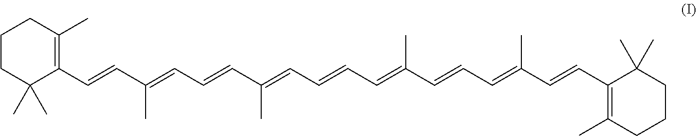 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]
wherein
a compound of formula (II) 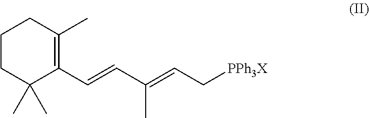
wherein
X is halogen
is reacted with a compound of formula (III) 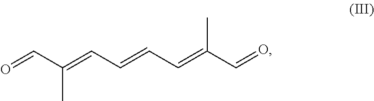
wherein at least one compound of formula (IV) 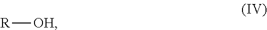
wherein
R is a linear or branched C₃-C₁₀alkyl moiety
is used as the solvent.

2. Process according to claim 1, wherein a compound of formula (II),
wherein
X is Cl, Br or I,
is used.

3. Process according to claim 1, wherein the compound of formula (IIa) 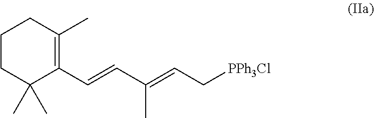
is used.

4. Process according to claim 1, wherein the process is carried out in at least one alcohol of formula (IV), wherein
R is a linear or branched C₄-C₈alkyl moiety.

5. Process according to claim 1, wherein the process is carried out in at least one alcohol of formula (IV), wherein
R is a linear or branched C₅alkyl moiety.

6. Process according to claim 1, wherein the process is carried out in 1-pentanol, 2-pentanol, 2-methyl-butan-1-ol, 2-methyl-2-butanol and/or 3-methyl-1-butanol.

7. Process according to claim 1, wherein the process is carried out in the presence of at least one base.

8. Process according to claim 7, wherein the base is an alkali hydroxide, earth alkali hydroxide, alkali carbonate, earth alkali carbonate, KF/Al₂O₃, NaOCH₃ and KOCH₃.

9. Process according to claim 7, wherein the base is chosen from the group consisting of CsOH, KOH, NaOH, Na₂CO₃ and KCO₃.

10. Process according to claim 7, wherein the at least base is used in an amount of 1.5 to 4 mol-equivalent (in view of the compound of formula (II).

11. Process according to claim 1, wherein the compound of formula (II) is used in an amount of at least 2 mol-equivalent (in view of the compound of formula (III).

12. Process according to claim 1, wherein process according to the present invention is usually carried out a temperature of −5° C. to 120° C.
